(19) United States
(12) Patent Application Publication
(10) Pub. No.: US 2007/0072836 A1
(43) Pub. Date: Mar. 29, 2007

(54) Solid peroral contraceptive preparations

(76) Inventors: Sabine Fricke, Jena (DE); Hagen Gericke, Jena (DE); Ralf Ladwig, Jena (DE); Alexander Buske, Jena (DE); Harald Raethe, Arnsgereuth (DE)

Correspondence Address: STRIKER, STRIKER & STENBY, 103 EAST NECK ROAD, HUNTINGTON, NY, 11743, US

(21) Appl. No.: 11/352,898

(22) Filed: Feb. 13, 2006

Related U.S. Application Data
(60) Provisional application No. 60/653,182, filed on Feb. 15, 2005.
(60) Provisional application No. 60/653,183, filed on Feb. 15, 2005.

(51) Int. Cl. A61K 31/57

(52) U.S. Cl. 514/170

(57) ABSTRACT

The solid peroral contraceptive contains an effective ingredient combination of dienogest in a daily dosage of equal to or less than 2.0 mg and ethinyl estradiol in a daily dosage of less than 0.03 mg together with one or more pharmaceutically acceptable carriers. The dienogest is released in two stages, while the ethinyl estradiol is released with the first stage portion of the dienogest.

### CROSS-REFERENCE

U.S. Provisional Application No. 60/653,182, filed Feb. 15, 2005, also discloses the invention described and claimed herein and provides a basis for a claim of priority for that invention under 35 U.S.C. 119.

### BACKGROUND OF THE INVENTION

1. The Field of the Invention

The subject matter of the present invention is a contraceptive preparation or drug, which contains less than or equal to 2 mg of 17α-cyanomethyl-17β-hydroxyestra4, 9-dien-3-one (dienogest) and less than 0.030 mg of 17α-ethinyl estradiol (ethinyl estradiol) and which releases dienogest in two stages.

2. Related Art

Oral contraceptives comprising a gestagen ingredient and an estrogen ingredient were first marketed more than 60 years ago. Three essential properties characterize the “contraceptive pill”: contraceptive reliability, very good cycle control and a minimum of side effects.

Since the introduction of hormonal contraceptives research has been directed to the development of contraceptive preparations, which reduce undesirable side effects while simultaneously providing good contraceptive reliability and cycle control. These undesirable side effects include arterial and venous thromboses and influences on carbohydrate and lipometabolism, which are caused by a higher gestagen and estrogen content than necessary for contraceptive action. WO 98/004269 discloses, among other things, oral administration of a combination of 250 μg to 4 mg of dienogest and 10 μg to 20 μg of ethinyl estradiol for contraception. In order to achieve a substantial: reduction of the total amount of steroids administered per cycle, while maintaining good cycle control, a low dosage gestagen/estrogen combination is administered for 23 to 25 days of the 28-day menstruation cycle. However no results are disclosed in this patent, which show that the inventive concept is successful.

WO 01/015701 claims a pharmaceutical composition for oral administration during 21 days of a 28-day menstruation cycle, which contains drospirenone and ethinyl estradiol, also in a low dosage, in which the drospirenone is present in micronized form. A rapid release of the steroids is especially notable.

EP 0 803 250 discloses a pharmaceutical tablet, which has a pharmacologically effective tablet core and an outer sugar coating, which can contain, among other ingredients, dienogest and ethinyl estradiol. Its desired rapid release action is influenced by microcrystalline cellulose.

It is also known that low dosage oral contraceptives should really be taken at daily equal time intervals. If this condition is not observed the effective concentration required for oral contraception is not attained, i.e. the effective concentration can reach values below the minimum concentration necessary for effective contraception,—and oral contraception is not guaranteed. That means that the user is asked to be very aware of the administration cycle and follow it with great care.

### SUMMARY OF THE INVENTION

It is an object of the present invention to provide a solid peroral contraceptive preparation, in which the conventionally used amount of the effective ingredient combination of dienogest and ethinyl estradiol is reduced, but the contraceptive action of the conventional preparation is reliably maintained.

This object and others which will be made more apparent hereinafter are attained by a solid peroral contraceptive preparation containing less than or equal to 2.0 mg of dienogest and less than 0.030 mg of ethinyl estradiol, which releases dienogest proportionately in two stages.

Embodiments of the solid peroral contraceptive preparation according to the invention contain 1.5 mg to 2 mg of dienogest and 0.015 mg to 0.020 mg of ethinyl estradiol.

In the contraceptive preparation according to the invention the dienogest has a rapid in-vitro release within a first stage and a delayed in-vitro release within a second stage. The ethinyl estradiol has a conventional rapid in-vitro release.

The portion of the dienogest released in the second stage amounts to at least 5%, preferably greater than 30%, as determined with a dissolution test according to Ph. Eur. performed by means of a rotating bottle apparatus using 1000 ml of water at 37° C. as dissolution medium and with stirring at stirring speed of 50 rpm.

In the second stage 10% to 90% of the dosage of dienogest can be released after 180 to 360 minutes.

In the first stage the in-vitro release of the dosage of ethinyl estradiol and up to 75% of the dosage of dienogest occurs in a maximum of 45 minutes, preferably up to 70% in 30 minutes, as determined with the above-indicated dissolution test.

One embodiment of the solid peroral contraceptive preparation according to the invention is a film tablet with a tablet core containing dienogest of the second stage and a film coating containing dienogest of the first stage and the entire amount of ethinyl estradiol.

It was found that ascorbic acid stabilizes ethinyl estradiol as soon as it is added to the effective-ingredient-containing film coating. The ascorbic acid content amounts to from 0.02 to 1.0%, preferably from 0.025 to 0.25%.

The effective ingredient release of the delayed fraction of dienogest can be controlled by a number of retardation principles. Examples of these retardation principles are embodied in inert plastic matrices, hydrocolloids, ion exchangers, retarding jackets, stomach acid resistant coatings, pellet mixtures, mixtures of minitablets and/or granulates, microcapsules, osmotic controlled systems and erosion-controlled systems, diffusion-controlled systems and their combinations and fat- and wax-containing matrices.

The tablets described in the following examples are master models. Other tablet embodiments, such as oblong tablets and tablets which influence erosion behavior of hydrophilic matrices are conceivable.

It was surprisingly found that the partially delayed release of dienogest from the contraceptive preparation permits the use of lower dosages of the effective ingredient combination of dienogest and ethinyl estradiol than are used in conventional oral contraceptives containing dienogest and ethinyl estradiol. Reliability is guaranteed without requiring that the contraceptive preparation must really unconditionally be taken at equal daily time intervals.

The number of daily dosage units of the contraceptive preparation according to suitable embodiments of the present invention can be 21, 22, 23, 24 or 25 daily dosage units and the number of effective ingredient free daily dosage units can amount to 7, 6, 5, 4 or 3 daily dosage units in a 28-day menstrual cycle.

Other embodiments in which the total number of daily dosage units is 28 or a multiple of 28, for example 2 to 3 times 28, which contain less than or equal to 2.0 mg of dienogest and less than 0.030 mg of dienogest, are possible.

Also other embodiments with gestagens, such as levonorgestrel, gestoden and others and/or estradiol valerate, which are suitable also for hormone replacement (also sequential) besides contraception, are possible.

The embodiments shown in the examples may be varied in regard to their dosage and still be within the metes and bounds of the present invention. The fraction of dienogest of the second retarded stage should preferably be greater than 30% of the total dienogest dosage. That includes embodiments with 35%, 40%, 45%, 50% or 55%, but also 70%, 75%, 80% of the total dienogest dosage. The time, in which the fraction of dienogest of the second retarded stage is completely released, varies in the examples from 180 min to 360 min. However shorter or longer release times may be attained by variation of the times shown in the examples.

Release times in whole hours, for example 1 h, 2 h, 3 h, 4 h, also 6 h, 7 h by means of the conventional variation range of the effective ingredient release.

It has been shown that the effective ingredient combination in the pharmaceutical preparation according to the present invention has anti-androgenic properties besides contraceptive action. Thus this preparation can be used for prophylaxis and therapy of androgen-induced conditions, especially acne.

### BRIEF DESCRIPTION OF THE DRAWING

The objects, features, and advantages of the invention will now be illustrated in more detail with the aid of the following description of the preferred embodiments, with reference to the accompanying figures in which:

FIG. 1 is a graphical illustration of respective release profiles (prior art) for dienogest and ethinyl estradiol from a conventional Valette® tablet preparation containing 2 mg of dienogest and 0.030 mg of ethinyl estradiol;

FIG. 2 is a graphical illustration of respective release profiles for dienogest and ethinyl estradiol from two embodiments of the film tablet according to the invention, which were made and measured according to examples 2 and 3 respectively, and which each contain 2 mg of dienogest and 0.020 mg of ethinyl estradiol;

FIG. 3 is a graphical illustration of respective release profiles for dienogest and ethinyl estradiol from four embodiments of the film tablet according to the invention, which were made and measured according to example 8 hereinbelow, and which each contain 1.5 mg of dienogest and 0.015 mg of ethinyl estradiol; and

FIG. 4 is a graphical illustration of respective release profiles for dienogest and ethinyl estradiol from an additional embodiment of the film tablet according to the invention, which was made and measured according to example 8 hereinbelow.

### EXAMPLES

### Example 1

Valette® is a conventional sugar-coated tablet for oral contraception containing 0.030 mg ethinyl estradiol and 2.0 mg dienogest in a tablet core, which is coated with sugar-containing jacket.

Measurement of the Release Profiles

Dissolution test was performed according to Ph. Eur., 4^{th} Edition, Main Work 2002, 2.9.3., flat-paddle stirring apparatus, 50 rpm, dissolution medium, 1000 ml water.

Measurement of the amount of dienogest and ethinyl estradiol released by means of high-pressure liquid chromatography.

FIG. 1 shows a typical prior art release profile of this sort of contraceptive containing a combination of gestagen and estrogen. A release behavior of at least 75% of the effective ingredient dosage within 45 minutes, preferably of 70% within 30 minutes, is designated a rapid release.

### Example 2

2 mg of dienogest and 0.02 mg ethinyl estradiol, wherein 1 mg of dienogest and 0.02 mg ethinyl estradiol are released rapidly and 1 mg of dienogest is released in a delayed manner according to the invention.

Description

This example describes a film tablet with a matrix core. The core of the film tablet contains 1 mg dienogest in a hydrophilic erosion matrix with a metolose base ingredient. The matrix provides a retarded dienogest release. The tablet core was coated with a rapidly dissolving film, which contains 1.0 mg dienogest and 0.02 mg ethinyl estradiol. The film tablet was also coated with an additional rapidly dissolving paint layer containing iron oxide pigments for light protection.

The tablet composition and manufacture are explained below in connection with the following Table I.

Manufacture

Granulate 1: Dissolve povidone in ethanol, granulate the other substances listed above under “Granulate 1” in a fluidized-bed granulator.

Granulate 2: Dissolve maltodextrin in water, granulate the other substances listed under “Granulate 2” in a fluidized-bed granulator.

Granulate 1, granulate 2 and the outer phase are mixed in a container mixer to form a mixture.

Make a tablet from the mixture (136 mg, oblong stamp 4.0×10.0 mm, bulge, 4.5 mm).

Film 1: coat tablets formed with an aqueous suspension of substances listed above under “film 1” to form film tablets.

Film 2: coat the film tablets with an aqueous suspension of substances listed above under “film 2” to form the final effective-ingredient-containing film tablets.

TABLE I
TABLET COMPOSITION - EXAMPLE 2
Tablet Core

| Granulate 1 | | |
|---|---|---|
| Dienogest | 1.0000 | mg |
| Metolose | 7.5000 | mg |
| 90SH-4000 | | |
| Lactose | 21.0000 | mg |
| monohydrate | | |
| Corn starch | 14.0000 | mg |
| Povidone K25 | 1.5000 | mg |
| (10% in ethanol) | | |
| Granulate 2 | | |
| Lactose | 54.0000 | mg |
| monohydrate | | |
| Corn Starch | 27.1000 | mg |
| Maltodextin (25% in water) | 6.9000 | mg |
| Outer Phase | | |
| Carboxymethylstarch | 1.500 | mg |
| sodium | | |
| Magnesium stearate | 1.500 | mg |

Film Coating

| | | |
|---|---|---|
| Effective Ingredient | | |
| Film 1 | | |
| Dienogest | 1.0000 | mg |
| Ethinyl estradiol | 0.020 | mg |
| Methocel 5 | 2.250 | mg |
| Talcum | 0.450 | mg |
| Titanium dioxide | 0.280 | mg |
| Colored Film 2 | | |
| Methocel 5 | 3.375 | mg |
| Talcum | 0.675 | mg |
| Titanium dioxide | 1.875 | mg |
| Iron oxide, red | 0.075 | mg |

Measurement of Release Profiles

A dissolution test for the film tablets prepared above was performed according to Ph. Eur., 4^{th} Edition, Main Work 2002, 2.9.3., flat-paddle stirring apparatus, 50 rpm, dissolution medium, 1000 ml water.

measurement of amounts of dienogest and ethinyl estradiol released were made by means of high-pressure liquid chromatography.

### Example 3

The film tablet of example 3 contained 2 mg of dienogest and 0.02 mg ethinyl estradiol, wherein 1 mg of dienogest and 0.02 mg ethinyl estradiol are released rapidly and 1 mg of dienogest is released in a delayed manner.

Description

This example describes a film tablet with a matrix core. The core of the film tablet contains 1 mg dienogest in a hydrophilic erosion matrix with a metolose base ingredient. The matrix provides a retarded dienogest release. To avoid interaction between the retarding core and the effective-ingredient-containing film the core was coated with a blocking layer, before the effective-ingredient-containing film was applied. The tablet core was coated with a rapidly dissolving film, which contains 1.0 mg of dienogest and 0.02 mg of ethinyl estradiol. The film tablet was also coated with an additional rapidly dissolving paint layer containing iron oxide pigments for light protection.

The tablet composition and manufacture are explained below in connection with the following Table II.

TABLE II
TABLET COMPOSITION - EXAMPLE 3
Tablet Core

| Granulate | | |
|---|---|---|
| Dienogest | 1.0000 | mg |
| Metolose | 7.5000 | mg |
| 90SH-4000 | | |
| Lactose | 31.0000 | mg |
| monohydrate | | |
| Corn starch | 24.0000 | mg |
| Povidone K25 (10% | 2.0000 | mg |
| in ethanol) | | |
| Outer Phase | | |
| Tabletose | 21.000 | mg |
| Avicel PH 102 | 16.200 | mg |
| Magnesium stearate | 1.500 | mg |

Film Coating

| | | |
|---|---|---|
| Blocking layer | | |
| Opadry AMB white ® | 7.0000 | mg |
| Comprising: | | |
| PVP part. hydrolized | | |
| Titanium dioxide | | |
| Soyalecithin | | |
| Xanthan | | |
| Effective Ingredient | | |
| Film 1 | | |
| Dienogest | 1.000 | mg |
| Ethinyl estradiol | 0.020 | mg |
| Methocel 5 | 2.250 | mg |
| Talcum | 0.430 | mg |
| Titanium dioxide | 0.280 | mg |
| Iron oxide, red | 0.020 | mg |

Manufacture

Granulate: Dissolve povidone in ethanol; granulate the other substances listed above under “Granulate” in a fluidized-bed granulator. Granulate and the outer phase are mixed in a container mixer to form a mixture.

Make tablets from the mixture (104 mg, oblong stamp 4.0×10.0 mm, bulge, 4.5 mm).

Blocking layer: coat tablets formed with an aqueous suspension of substances listed above under “blocking layer” to form film tablets.

Effective-ingredient-containing film 1: coat the film tablets with an aqueous suspension of substances listed above under “film 1” to form the final effective-ingredient-containing film tablets.

Measurement of Release Profiles

A dissolution test for the film tablets prepared above was performed according to Ph. Eur., 4^{th} Edition, Main Work 2002, 2.9.3., flat-paddle stirring apparatus, 50 rpm, dissolution medium, 1000 ml water.

measurement of amounts of dienogest and ethinyl estradiol released were made by means of high-pressure liquid chromatography.

The film tablets of examples 2 and 3 differ in the structure of their film jackets. The effective-ingredient-containing film releases the entire ethinyl estradiol dosage and the rapid release portion of the dienogest dosage. Similarly the colored film in example 2 dissolves rapidly. The blocking layer in example 3 prevents interaction between the effective-ingredient-containing film and the core and dissolves slower than the effective-ingredient-containing film. Both film tablets have a matrix core, comprising a hydrophilic erosion matrix. This erosion matrix slowly releases the retarded portion of the dienogest dosage.

FIG. 2 shows the measured release profiles for the tablets of examples 2 and 3. The film tablets of both examples release about 80% of the ethinyl estradiol dosage within 45 minutes and about 50% of the dienogest dosage within 45 minutes. The remaining portions of the die nogest dosages in the film tablets are released within about 360 min in the case of example 2 and within 180 min in the case of example 3.

### Example 4

In example 4 the release of the retarded portion of dienogest is controlled with a lipophilic matrix.

Description

This example describes a tablet containing 2.0 mg dienogest and 0.020 mg ethinyl estradiol and operates on a lipophilic retardation principle. 1 mg of dienogest is embedded in a lipophilic matrix by spraying. The rapidly released portion of the dienogest dosage and the ethinyl estradiol are mixed in this matrix.

The tablet composition and manufacture are explained below in connection with the following Table III.

Manufacture

dissolve the-retarded portion of dienogest and cetylstearyl alcohol in ethanol at 50° C.

spray dienogest/cetylstearyl alcohol solution into lactose and corn starch in a fluidized-bed granulate and dry.

dissolve maltodextrin in water.

mix Na-carboxymethyl starch and magnesium stearate.

tablet to form tablets with a diameter of 5.5 mm with a mass of 90 mg.

TABLE III
TABLET COMPOSITION - EXAMPLE 4

| | |
|---|---|
| Dienogest (ethanolic solution) | 1.00 mg |
| Cetylstearyl alcohol (ethanolic | 9.00 mg |
| solution) | |
| Lactose monohydrate | 57.98 mg |
| Corn starch | 10.00 mg |
| Dienogest | 1.00 mg |
| Ethinyl estradiol | 0.02 mg |
| Maltodextrin (20% solution in | 9.00 mg |
| water) | |
| Na-carboxymethyl starch | 1.00 mg |
| Magnesium stearate | 1.00 mg |

### Example 5

In example 5 the release of the retarded portion of dienogest is controlled by using different grain sizes.

Description

This example 5 describes a tablet, in which the dienogest has different grain fractions. The desired adjustment or setting of the particles sizes occurs by means of fractional crystallization.

The tablet composition and manufacture are explained below in connection with the following Table IV.

TABLE IV
TABLET COMPOSITION - EXAMPLE 5

| | | |
|---|---|---|
| Dienogest (average grain size 3 μm) | 0.667 | mg |
| Dienogest (average grain size 180 μm) | 0.667 | mg |
| Dienogest (average grain size 270 μm) | 0.667 | mg |
| Ethinyl estradiol | 0.02 | mg |
| Lactose monohydrate | 47.18 | mg |
| Maltodextrin (20% solution in water) | 9.00 | mg |
| Corn starch | 25.00 | mg |
| Magnesium stearate | 0.80 | mg |

Manufacture

granulate the above-listed substances except for magnesium stearate with the maltodextrin solution.

add magnesium stearate to form a mixture.

tablet the mixture (tablets 5.5 mm diameter, 80. mg mass).

### Example 6

A chemical problem is connected with the reduction of the ethinyl estradiol dosage. Increasing dilution of the effective ingredient in the contraceptive preparation accelerates its chemical decomposition during storage, perhaps already during manufacture of the contraceptive preparation. Surprisingly ascorbic acid acts as an effective stabilizer for ethinyl estradiol. Example 6 shows that ascorbic acid is an effective stabilizer in the exemplary formulation.

Description

The example describes a stabilizing effect of ascorbic acid on ethinyl estradiol with the aid of exemplary formulations in a stress test.

The compositions of the exemplary preparations are tabulated in Table V below.

TABLE V
PREPARATION COMPOSITION - EXAMPLE 6

| | Variant | | | | |
|---|---|---|---|---|---|
| | 6.1 | 6.2 | 6.3 | 6.4 | 6.5 |
| Ascorbic | — | 0.02 mg | 0.20 mg | — | — |
| Acid, | | | | | |
| Binding | | | | | |
| agent | | | | | |
| solution | | | | | |
| Ascorbic | — | — | — | 0.02 mg | 0.20 mg |
| Acid, | | | | | |
| In mixture | | | | | |
| Dienogest | 2.00 mg | 2.00 mg | 2.00 mg | 2.00 mg | 2.00 mg |
| Ethinyl | 0.02 mg | 0.02 mg | 0.02 mg | 0.02 mg | 0.02 mg |
| estradiol | | | | | |
| Lactose | 47.18 mg | 47.16 mg | 46.98 mg | 47.16 mg | 46.98 mg |
| monohydrate | | | | | |
| Corn starch | 24.00 mg | 24.00 mg | 24.00 mg | 24.00 mg | 24.00 mg |
| Maltodextrin | 6.00 mg | 6.00 mg | 6.00 mg | 6.00 mg | 6.00 mg |
| Magnesium | 0.80 mg | 0.80 mg | 0.80 mg | 0.80 mg | 0.80 mg |
| stearate | | | | | |

Manufacture

mix lactose, corn starch and dienogest in granulator, spray with a solution of ethinyl estradiol in ethanol and dry.

mix with a binding agent solution of maltodextrin in water, granulate, dry and mix with magnesium stearate to form a mixture.

tablet to form tablets of 80 mg mass and 5.5 mm diameter from the mixture.

Testing the Content in Stress Tests

The tablets were stored in an open container at 60° C. and 80% relative humidity. After storage time of 42 days the tablets were removed and tested. The measurement of the content of ethinyl estradiol occurred by means of HPLC and is related to the content of the starting material. The results are shown below in Table VI.

TABLE VI
Stress Test Results - Variation of Ethinyl Estradiol
Content with Storage Time

| | Variant | | | | |
|---|---|---|---|---|---|
| | 6.1 | 6.2 | 6.3 | 6.4 | 6.5 |
| Ascorbic Acid | — | 0.02 mg | 0.20 mg | — | — |
| in Binding | | | | | |
| Agent | | | | | |
| Ascorbic Acid | — | — | — | 0.02 mg | 0.20 mg |
| in Mixture | | | | | |
| Content after | 79.1% | 90.1% | 91.9% | 91.6% | 96.5% |
| 42 days | | | | | |

### Example 7

Description

In this example the stabilization of ethinyl estradiol in the effective-ingredient-containing layer of the film tablets is described. The tablet composition is outlined in the Table-VII listed herein below.

TABLE VIII
FILM TABLET COMPOSITION - EXAMPLE 7

| | | |
|---|---|---|
| CORE | 104 | mg |
| Dienogest | 0.750 | mg |
| Metolose 90SH-4000 | 7.500 | mg |
| Lactose monohydrate | 45.250 | mg |
| Corn starch | 10.000 | mg |
| Povidone K25 (10% in water) | 2.000 | mg |
| Tablettose | 30.000 | mg |
| Avicel PH 102 | 7.200 | mg |
| Magnesium Stearate | 1.300 | mg |
| Blocking Layer | 6 | mg |
| Eudragit RL 30 D (for lacquer drying) | 3.500 | mg |
| Macrogol 6000 | 0.700 | mg |
| Talcum | 1.800 | mg |
| Effective-ingredient-containing film | 3 | mg |
| Dienogest | 0.750 | mg |
| Ethinyl estradiol | 0.015 | mg |
| Ascorbic acid | 0.200 | mg |
| Methocel 5 | 1.6875 | mg |
| Talcum | 0.2375 | mg |
| Titanium dioxide | 0.210 | mg |
| Colored layer | 3 | mg |
| Methocel 5 | 1.500 | mg |
| PEG 6000 | 0.300 | mg |
| Talcum | 0.300 | mg |
| Titanium dioxide | 0.850 | mg |
| Iron oxide, red | 0.050 | mg |

Manufacture

Granulate dienogest, metolose 90SH-4000, lactose monohydrate and corn starch with, the aqueous povidone K25 and/or maltodextrin solution.

Add the tablettose, Avicel PH 102 and magnesium stearate to the dried granulate to form a mixture.

Tablet the mixture to form tablets.

Coat the resulting tablets with the appropriate films in a drum coater.

### Example 8

Description

Five variants 8.1 to 8.5 of film tablets each with a total dosage of 1.5 mg of dienogest and 0.015 mg of ethinyl estradiol are described. The film tablets comprise a release-retarding matrix core and a rapidly dissolving film jacket as well as a colored layer. In two variants 8.1 and 8.2 a blocking layer is provided between the tablet core and the effective-ingredient-containing film.

In the five variants the retarded release fraction of the dienogest dosage was varied in a range from 33% to 66%. The recipe for the core was adjusted to obtain the desired release profile. The measured release profiles for variants 8.1 to 8.4 are illustrated graphically in FIG. 3. The release profile for variant 8.5 is shown in FIG. 4.

The tablet composition was outlined in the Table IX below.

TABLE IX
FILM TABLET COMPOSITION - EXAMPLE 8

| | Varient | | | | |
|---|---|---|---|---|---|
| | 8.1 | 8.2 | 8.3 | 8.4 | 8.5 |
| CORE | 104 mg | 104 mg | 104 mg | 104 mg | |
| Dienogest | 0.750 mg | 0.500 mg | 1.000 mg | 0.750 mg | 0.675 mg |
| Metolose 90SH-4000 | 7.500 mg | 7.500 mg | 7.500 mg | 9.000 mg | 9.000 mg |
| Lactose monohydrate | 45.250 mg | 39.500 mg | 45.000 mg | 29.750 mg | 42.925 mg |
| Corn starch | 10.000 mg | 10.000 mg | 10.000 mg | 24.000 mg | 15.000 mg |
| Povidone K25 (10% in | 2.000 mg | — | 2.000 mg | 2.000 mg | — |
| water) | | | | | |
| Maltodextrin (25% in water) | — | 8.000 mg | — | — | 6.000 mg |
| Tablettose | 30.000 mg | 30.000 mg | 30.000 mg | 21.000 mg | 8.500 mg |
| Avicel PH 102 | 7.200 mg | 7.200 mg | 7.200 mg | 16.200 mg | 7.000 mg |
| Magnesium Stearate | 1.300 mg | 1.300 mg | 1.300 mg | 1.300 mg | 0.900 mg |
| Blocking Layer | 6 mg | 6 mg | — | — | — |
| Eudragit RL 30 D (for | 3.500 mg | 3.500 mg | | | |
| lacquer drying) | | | | | |
| Macrogol 6000 | 0.700 mg | 0.700 mg | | | |
| Talcum | 1.800 mg | 1.800 mg | | | |
| Effective-ingredient- | 3 mg | 3 mg | 3 mg | 3 mg | |
| containing film | | | | | |
| Dienogest | 0.750 mg | 1.000 mg | 0.500 mg | 0.750 mg | 0.825 mg |
| Ethinyl estradiol | 0.015 mg | 0.015 mg | 0.015 mg | 0.015 mg | 0.015 mg |
| Methocel 5 | 1.6875 mg | 1.4987 mg | 1.8848 mg | 1.6875 mg | 4.060 mg |
| Talcum | 0.3375 mg | 0.2998 mg | 0.3700 mg | 0.3375 mg | 0.836 mg |
| Titanium dioxide | 0.210 mg | 0.1865 mg | 0.2302 mg | 0.210 mg | 0.264 mg |
| Colored layer | 3 mg | 3 mg | 3 mg | 3 mg | 3 mg |
| Methocel 5 | 1.500 mg | 1.500 mg | 1.500 mg | 1.500 mg | 1.500 mg |
| PEG 6000 | 0.300 mg | 0.300 mg | 0.300 mg | 0.300 mg | 0.300 mg |
| Talcum | 0.300 mg | 0.300 mg | 0.300 mg | 0.300 mg | 0.300 mg |
| Titanium dioxide | 0.850 mg | 0.850 mg | 0.850 mg | 0.850 mg | 0.850 mg |
| Iron oxide, red | 0.050 mg | 0.050 mg | 0.050 mg | 0.050 mg | 0.050 mg |

Manufacture

Granulate the dienogest, metolose 90SH-4000, lactose monohydrate and corn starch with aqueous povidone K25 and/or maltodextrin solution.

Add tablettose, avicel PH 102 and magnesium stearate to the dried granulate to form a mixture.

Tablet the mixture.

Coat the tablets with the appropriate film in a drum coater.

Measurement of Release Profiles

A dissolution test for the film tablets prepared above was performed according to Ph. Eur., 4^{th} Edition, Main Work 2002, 2.9.3., flat-paddle stirring apparatus, 50 rpm, dissolution medium, 1000 ml water.

Measurement of amounts of dienogest and ethinyl estradiol released were made by means of high-pressure liquid chromatography.

The measured profiles for the five variants 8.1 to 8.5 are shown in FIGS. 3 and 4.

### Example 9

Analogous to the variants disclosed in example 8 variants of film tablets with a total dosage of 2.0 mg dienogest and 0.015 mg of ethinyl estradiol were described, prepared and tested.

The contraceptive action of preparations, which contain dienogest and ethinyl estradiol, may be tested in various investigations, for example in a randomized open clinical study. Different laboratory and diagnostic studies were performed. FSH, LH, estradiol, progesterone, “spinability” and fern phenomenon. Follicle maturation was tested with the aid of ultrasonic techniques. Furthermore SHBG, CBG, total testosterone, triglyceride, HDL cholesterol, HDL cholesterol, serum glucose as well as blood pressure, heart rate, body weight and bleeding behavior were recorded.

In the following claims dienogest means 17α-cyanomethyl-17β-hydroxy-estra-4, 9-dien-3-one and ethinyl estradiol means 17α-ethinyl estradiol.

Unless otherwise indicated, all percentages are percentages by weight.

While the invention has been illustrated and described as embodied in a solid peroral contraceptive preparation, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A solid peroral preparation for contraception, said preparation containing dienogest in an amount that is less than or equal to 2.0 mg and ethinyl estradiol in an amount that is less than 0.030 mg, wherein said dienogest is released in at least two stages and at least one of said stages is delayed in comparison to another of said stages.

2. The solid peroral preparation as defined in claim 1, containing one portion of said dienogest that is released in a comparatively delayed manner and another portion of said dienogest that is released in a comparatively not delayed or rapid manner.

3. The solid peroral preparation as defined in claim 1, containing one portion of said dienogest that is released in a comparatively delayed manner and another portion of said dienogest that is released in a comparatively not delayed or rapid manner as well as a total content of said ethinyl estradiol that is released in a comparatively not delayed manner.

4. The solid peroral preparation as defined in claim 1, comprising a film tablet, and wherein said film tablet consists of a tablet core and a coating; and
wherein said tablet core contains one portion of said dienogest that is released in a comparatively delayed manner; and
wherein said coating contains another portion of said die nogest that is released in a comparatively not delayed or rapid manner as well as a total content of said ethinyl estradiol that is released in a comparatively not delayed manner.

5. The solid peroral preparation as defined in claim 1, containing from 1.5 mg to 2.0 mg of said dienogest and from 0.015 mg to 0.020 mg of said ethinyl estradiol.

6. The solid peroral preparation as defined in claim 4, wherein said film tablet contains from 1.5 mg to 2.0 mg of said dienogest and from 0.015 mg to 0.020 mg of said ethinyl estradiol.

7. The solid peroral preparation as defined in claim 4, wherein at least 10% of said dienogest dissolves from said tablet core after more than 30 minutes from said tablet core in a dissolution test using water at 37° C. as dissolution medium and with stirring with a stirring speed of 50 rpm.

8. The solid peroral preparation as defined in claim 4, wherein at least 30% of said dienogest dissolves from said tablet core after more than 30 minutes from said tablet core in a dissolution test using water at 37° C. as dissolution medium and with stirring with a stirring speed of 50 rpm.

9. The solid peroral preparation as defined in claim 4, wherein said coating contains ascorbic acid as ethinyl estradiol stabilizer.

10. The solid peroral preparation as defined in claim 9, wherein said coating contains from 0.02 to 1.0% of said ascorbic acid.

11. The solid peroral preparation as defined in claim 9, wherein said coating contains from 0.025 to 0.25% of said ascorbic acid.

12. A contraceptive preparation comprising a plurality of daily dosage units each containing an effective ingredient combination of dienogest in an amount of equal to or less than 2.0 mg and ethinyl estradiol in an amount of less than 0.030 mg and another plurality of daily dosage units containing no effective ingredient;
wherein said plurality consists of 21, 22, 23, 25 or 25 of said daily dosage units containing said effective ingredient combination and said another plurality consists of 7, 6, 5, 4 or 3 of said daily dosage units containing no effective ingredient.

13. The contraceptive preparation as defined in claim 12, wherein each of said daily dosage units contains from 1.5 mg to 2.0 mg of said dienogest and from 0.015 mg to 0.020 mg of said ethinyl estradiol.

14. The contraceptive preparation as defined in claim 12, wherein each of said daily dosage units containing said effective ingredient combination consists of a film tablet for oral administration, said film tablet consists of a tablet core and a coating; and
wherein said tablet core contains one portion of said dienogest that is released in a comparatively delayed manner; and
wherein said coating contains another portion of said dienogest that is released in a comparatively not delayed or rapid manner as well as a total content of said ethinyl estradiol that is released in a comparatively not delayed manner.

15. The contraceptive preparation as defined in claim 14, wherein said coating contains ascorbic acid as ethinyl estradiol stabilizer.
